(19) United States
(12) Patent Application Publication
(10) Pub. No.: US 2005/0155109 A1
(43) Pub. Date: Jul. 14, 2005

(54) Tomato having reduced deoxyhypusine synthase activity caused by non-transgenic alterations in a deoxyhypusine synthase gene

(75) Inventors: Susan Hurst, Seattle, WA (US); Ann J. Slade, Kenmore, WA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Anawah Inc.

(21) Appl. No.: 10/990,637

(22) Filed: Nov. 17, 2004

Related U.S. Application Data
(60) Provisional application No. 60/520,792, filed on Nov. 17, 2003.
(60) Provisional application No. 60/563,920, filed on Apr. 21, 2004.
(60) Provisional application No. 60/622,406, filed on Oct. 26, 2004.

(51) Int. Cl. A01H 1/00; C12N 15/82; A01H 5/00; C12N 9/06

(52) U.S. Cl. 800/284; 800/317.4; 435/191

(57) ABSTRACT

A series of independent non-transgenic mutations found in at least one deoxyhypusine synthase gene of tomato; tomato plants having these mutations in one or more of their deoxyhypusine synthase genes; and a method of creating and identifying similar and/or additional mutations in a deoxyhypusine synthase gene by screening pooled and/or individual tomato plants. The tomato plants of the present invention exhibit altered deoxyhypusine synthase activity and delayed post harvest softening of their tomato fruit without having the inclusion of foreign nucleic acids in their genomes. Novel nucleotide and protein sequences for deoxyhypusine synthases in tomato and their uses.

### FIELD OF THE INVENTION

This invention concerns non-transgenic mutations in a deoxyhypusine synthase gene of tomato and tomato plants having these non-transgenic mutations in at least one of their deoxyhypusine synthase genes. This invention further concerns tomato plants having delayed post harvest softening of their fruits as a result of non-transgenic mutations in at least one of their deoxyhypusine synthase genes. This invention further concerns a method of creating non-transgenic tomato plants exhibiting an alteration in deoxyhypusine synthase activity. In addition, this invention concerns novel nucleotide sequences and a novel protein sequence for deoxyhypusine synthases identified in tomato and the use of these sequences to modify senescence in tomatoes and tomato plants.

### BACKGROUND

One of the main challenges facing the fresh tomato industry is to deliver tomato fruit to market that have been vine-ripened (i.e., thus desirable to consumers in taste, texture, and color) but remain firm without the usual softening that accompanies the onset of senescence in harvested fruit. Because traditional breeding methods are very labor intensive and could take years to develop a novel tomato variety that may display only a modest increase in shelf life, recent studies have utilized genetic and biochemical techniques in an effort to elucidate the factors that regulate senescence. By identifying and modifying the expression of specific genes that control cell death and senescence in plants, researchers and breeders hope to develop new tomato varieties that have the desirable qualities of vine-ripened fruit but are resistant to post harvest softening and display a longer-shelf life with reduced spoilage.

Recent data indicate deoxyhypusine synthase (DHS) and eukaryotic translation initiation factor 5A (eIF-5A) are important regulators of senescence in plants and animals. DHS is an enzyme that converts the inactive form of eIF-5A to its active form, hypusine-modified eIF-5A. By adding butylamine to a conserved lysine of eIF-5A, DHS catalyzes the formation of deoxyhypusine which is subsequently converted to hypusine by the enzyme deoxyhypusine hydoxylase. This hypusine-modified eIF5A plays an important role in cell growth and differentiation. It has been suggested that eIF-5A, localized to the nuclear pore, directs the translocation of specific mRNAs from the nucleus to the cytoplasm thereby facilitating the translation of particular proteins.

Studies in tomato (Wang et al., Journal of Biological Chemistry, 276(20): 17541-17549, 2001) revealed that messenger RNAs for DHS and eIF-5A are increased in senescing flowers and fruit and in leaves from plants that have been environmentally stressed (e.g., osmotic and temperature stress). The observation that DHS protein levels are also increased at the onset of senescence together with evidence that DHS plays a role in eIF-5A activation suggests a role for DHS and eIF-5A in the senescent process in plants. With these findings in mind, it has been postulated that eIF-5A facilitates the translation of the subset of genes activated at the onset of senescence. Together these data suggest that inhibition of DHS and/or eIF-5A in tomato may result in tomatoes with less senescence-related softening after harvest.

Genetic modification of DHS in tomato plants confirms the importance of this target protein to the commercial tomato industry. Tomatoes from transgenic tomato plants that express an antisense DHS transgene to reduce endogenous DHS protein levels are slower to soften post harvest with delayed spoilage compared to wild type tomatoes (U.S. Pat. No. 6,538,182). Additionally, these transgenic tomato plants have increased plant biomass: both leaf size and plant size are increased in transgenic plants compared to wild type plants. Seed yield is also greater in transgenic plants. Similar results (increased biomass and seed yield) are also obtained in transgenic Arabidopsis plants that express an antisense DHS transgene.

Multiple isoforms of eIF-5A have been isolated in tobacco, yeast, humans, and chickens. Four eIF-5A isoforms have been identified in tomato (Wang et al., Journal of Biological Chemistry, 276(20): 17541-17549, 2001). Tomato isoforms display 89-92% homology at the amino acid level and 70-80% homology at the nucleotide levels. Messenger RNA for the isoform eIF-5A1 is upregulated as a consequence of natural and induced senescence in the tomato but it is not known whether expression of the other tomato eIF-5A isoforms also increases.

In yeast and humans, only a single isoform of DHS has been identified to date. Similarly, only a single isoform of tomato DHS has been isolated to date though the presence of multiple fragments following restriction enzyme digestions of tomato genomic DNA raised the possibility that multiple DHS isoforms may exist in tomato (Wang et al., Journal of Biological Chemistry, 276(20): 17541-17549, 2001). A genomic sequence, corresponding to the single published tomato DHS complete cds., has not been published.

Together these data indicate that modulation of DHS and/or eIF-5A levels affect senescence in tomatoes. Further, they suggest that mutations in these genes that lead to a reduction in protein levels or protein function may be used to modify the senescent process and thereby affect tomato shelf life; however, these types of mutant tomato lines have never been identified. In addition, such mutations may be important for the development of tomato varieties that are stress resistant. It would be useful to have cultivated tomato plants exhibiting these traits.

Traditional breeding methods are laborious and time consuming. In addition, undesirable characteristics are often transferred along with the desired traits when tomato plants are crossed in a traditional breeding program. Transgenic technology can be used to modify the expression of particular genes; however, public acceptance of genetically modified plants, particularly with respect to plants used for food, is low. Therefore, a cultivated tomato that is resistant to post harvest softening and has a longer shelf life but is not the result of genetic engineering would be useful.

### SUMMARY OF THE INVENTION

In one aspect, this invention includes a tomato plant, tomatoes, seeds, plant parts and progeny thereof having an alteration in deoxyhypusine synthase activity caused by a non-transgenic mutation in at least one deoxyhypusine synthase gene.

In another aspect, this invention includes a tomato plant containing a mutated deoxyhypusine synthase gene, as well as fruit, seeds, pollen, plant parts and progeny of that plant.

In another aspect, this invention includes food and food products incorporating tomato plants having an alteration in deoxyhypusine synthase activity caused by a non-transgenic mutation in at least one deoxyhypusine synthase gene.

In another aspect, this invention includes a method of creating tomato plants exhibiting an alteration in deoxyhypusine synthase activity, comprising the steps of: obtaining plant material from a desired cultivar of tomato plant; inducing point mutations in at least one deoxyhypusine synthase gene of the plant material by treating the plant material with a mutagen; growing the mutagenized plant material to produce tomato plants; isolating genomic DNA from the tomato plants or from progeny of the tomato plant; amplifying segments of a deoxyhypusine synthase gene from the genomic DNA of the tomato plants or the progeny of the tomato plant using PCR primers specific to a deoxyhypusine synthase gene or to the DNA sequences adjacent to a deoxyhypusine synthase gene; and detecting point mutations in a deoxyhypusine synthase gene of at least one tomato plant.

In a further aspect, this invention includes a tomato plant, fruit, seeds, pollen or plant parts created according to the method of the present invention as well as novel DHS nucleotide and protein sequences for tomato.

### BRIEF DESCRIPTION OF THE SEQUENCE LISTING

SEQ ID NO: 1 shows the complete cds. for Lycopersicon esculentum deoxyhypusine synthase GenBank Accession Nos. AF296077. Start site (atg) begins at nucleotide 54; the stop codon (tga) ends at nucleotide 1199.

SEQ ID NO: 2 shows a novel genomic sequence for Lycopersicon esculentum deoxyhypusine synthase (DHS1) starting with the ATG start site and ending with the stop codon of the coding region.

SEQ ID NO: 3 shows the protein encoded by SEQ ID NO: 1 (GenBank Accession Number AAG53641).

SEQ ID NO: 4 shows a novel partial genomic sequence for a probable second Lycopersicon esculentum deoxyhypusine synthase, DSH2.

SEQ ID NO: 5 shows the novel coding sequences corresponding to the partial genomic sequence for a probable Lycopersicon esculentum deoxyhypusine synthase 2 (DSH2) in SEQ ID NO: 4.

SEQ ID NO: 6 shows the novel polypeptide as translated from the probable Lycopersicon esculentum deoxyhypusine synthase 2 (DHS2) coding sequence shown in SEQ ID NO: 5.

SEQ ID NOs: 7-15 show DNA sequences of PCR primers used for determining the genomic sequences of tomato DHS 1 and DHS2.

SEQ ID NOs: 16-27 show the DNA sequences of DHS specific PCR primers used to identify the DHS mutants of the present invention.

### DETAILED DESCRIPTION OF PREFERRED EMBODIMENTS

The present invention describes: tomato plants exhibiting altered deoxyhypusine synthase enzyme activity without the inclusion of foreign nucleic acids in the tomato plants' genomes. The present invention further describes a series of independent non-transgenic mutations in a deoxyhypusine synthase gene of tomato; tomato plants having these mutations in the deoxyhypusine synthase gene thereof; and a method of creating and identifying similar and/or additional mutations in at least one deoxyhypusine synthase gene of tomato plants. Furthermore, the present invention describes tomato plants exhibiting delayed post harvest softening of their tomato fruits due to altered deoxyhypusine synthase activity without the inclusion of foreign nucleic acids in the tomato plants' genomes. Further, the present invention describes novel genomic DNA sequences for tomato deoxyhypusine synthase, called here DSH1 and DHS2, and a novel protein sequence for DHS2 as well as the use of these sequences to modify post harvest softening and senescence in tomato fruit and tomato plants.

In order to create and identify the deoxyhypusine synthase gene mutations and tomatoes of the present invention, a method known as TILLING® was utilized. See McCallum et al., Nature Biotechnology, 18: 455-457, 2000; McCallum et al., Plant Physiology, 123: 439-442, 2000; and U.S. Pat. Nos. 5,994,075 and 20040053236, all of which are incorporated herein by reference. In the basic TILLING® methodology, plant material, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest.

Any cultivar of tomato having at least one gene with substantial homology to SEQ ID NO: 2 or 4 may be used in the present invention. The homology between the DHS gene and SEQ ID NO: 2 or 4 may be as low as 60% provided that the homology in the conserved region of the gene is higher. One of skill in the art may prefer a tomato cultivar having commercial popularity or one having specific desired characteristics in which to create the deoxyhypusine synthase-mutated tomato plants. Alternatively, one of skill in the art may prefer a tomato cultivar having few polymorphisms, such as an in-bred cultivar, in order to facilitate screening for mutations within a deoxyhypusine synthase gene.

In one embodiment of the present invention, tomato seeds were mutagenized and then grown into M1 plants. The M1 plants were then allowed to self-pollinate and seeds from the M1 plant were grown into M2 plants, which were then screened for mutations in their deoxyhypusine synthase genes. An advantage of screening the M2 plants is that all somatic mutations correspond to the germline mutations. One of skill in the art would understand that a variety of tomato plant materials including, but not limited to, seeds, pollen, plant tissue or plant cells, may be mutagenized in order to create a deoxyhypusine synthase-mutated tomato plants of the present invention. However, the type of plant material mutagenized may affect when the plant DNA is screened for mutations. For example, when pollen is subjected to mutagenesis prior to pollination of a non-mutagenized plant, the seeds resulting from that pollination are grown into M1 plants. Every cell of the M1 plants will contain mutations created in the pollen, thus these M1 plants may then be screened for deoxyhypusine synthase gene mutations instead of waiting until the M2 generation.

Mutagens that create primarily point mutations and short deletions, insertions, transversions, and/or transitions (about 1 to about 5 nucleotides), such as chemical mutagens or radiation, may be used to create the mutations of the present invention. Mutagens conforming with the method of the present invention include, but are not limited to, ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino] acridine dihydrochloride (ICR-170), and formaldehyde. Spontaneous mutations in a deoxyhypusine synthase gene that may not have been directly caused by the mutagen can also be identified using the present invention.

Any method of plant DNA preparation known to those of skill in the art may be used to prepare the tomato plant DNA for deoxyhypusine synthase mutation screening. For example, see Chen & Ronald, Plant Molecular Biology Reporter, 17: 53-57, 1999; Stewart & Via, Bio Techniques, 14: 748-749, 1993. Additionally, several commercial kits are available, including kits from Qiagen (Valencia, Calif.) and Qbiogene (Carlsbad, Calif.).

Prepared DNA from individual tomato plants were then pooled in order to expedite screening for mutations in the deoxyhypusine synthase genes of the entire population of plants originating from the mutagenized plant tissue. The size of the pooled group is dependent upon the sensitivity of the screening method used. Preferably, groups of four or more individuals are pooled.

After the DNA samples were pooled, the pools were subjected to deoxyhypusine synthase gene-specific amplification techniques, such as Polymerase Chain Reaction (PCR). For a general overview of PCR, see PCR Protocols: A Guide to Methods and Applications (Inns, M., Gelfand, D., Sninsky, J., and White, T., eds.), Academic Press, San Diego, 1990. Any primer specific to the deoxyhypusine synthase genes or the sequences immediately adjacent to the deoxyhypusine synthase genes may be utilized to amplify the deoxyhypusine synthase genes within the pooled DNA sample. Preferably, the primer is designed to amplify the regions of the deoxyhypusine synthase gene where useful mutations are most likely to arise. Most preferably, the primer is designed to detect mutations in the coding region of the deoxyhypusine synthase gene. Additionally, it is preferable for the primer to avoid known polymorphic sites in order to ease screening for point mutations. To facilitate detection of PCR products on a gel, the PCR primer may be labeled using any conventional labeling method.

In the present invention, a genomic DNA sequence for a deoxyhypusine synthase gene was constructed. Based upon the deoxyhypusine synthase cDNA sequence GenBank accession number AF296077 (SEQ ID NO: 1), sets of primers (SEQ ID NOs: 7-15) were designed to amplify overlapping segments of genomic DNA. PCR products were sequenced and a continuous DNA sequence extending between a start codon to a stop codon was deduced by aligning these overlapping segments (SEQ ID NO: 2). A resulting 4,755 nucleotide sequence, called here DHS1, was 100% homologous to the published complete cds. for DHS (SEQ ID NO: 1) in discrete regions but these regions of high homology were interrupted by intervening stretches of novel sequences. The novel stretches were interpreted to represent intronic regions in the genomic DNA sequence for DHS. A second sequence (DHS2, SEQ ID NO: 3), 1256 nucleotides in length, was identified. This second sequence also contained discrete regions of high homology (89.5%) to the DHS complete cds. (SEQ ID NO: 1) with intervening stretches of novel sequences; however, these intervening stretches of novel sequence exhibited low homology to the intervening stretches contained within DSH1. Therefore, DHS2 was interpreted to represent a probable genomic sequence for a second Lycopersicon esculentum deoxyhypusine synthase. The protein encoded by SEQ ID NO: 1 (shown in SEQ ID NO: 3) is also encoded by SEQ ID NO: 2. The protein encoded by the probable Lycopersicon esculentum deoxyhypusine synthase 2 (SEQ ID NO: 6) shows 85.7% homology with SEQ ID NO: 3.

The assembled genomic sequences (SEQ ID NO: 2 and 4) were then used to design primers to detect mutations within the coding regions of one or more Lycopersicon esculentum deoxyhypusine synthase genes of mutagenized plants. Exemplary primers (SEQ ID NOs: 16-27) that proved useful in identifying useful mutations within a deoxyhypusine synthase gene sequence are shown below in Table 1.

| | | | SEQ |
|---|---|---|---|
| | | | ID |
| NAME | ID | SEQUENCE | NO |
| DSLX | PR-0349 | TCGGAAAATCTGTTCACCCTCACAAG | 16 |
| DSRX | PR-0391 | GCATAGCAGCGATTCAGTGCTTGACA | 17 |
| DSL3 | PR-0214 | TTGGGCAGTTTCTCGGGAAGTCA | 18 |
| DSR5 | PR-0215 | TGGTCGGAAGGACAGCTTCCTCAA | 19 |
| DSL0 | PR-0211 | CGGCGGCAGTCTTGTTCCGTA | 20 |
| DSR6 | PR-0343 | TGAAACCCAAATATTGTCGGTGTAAAGCA | 21 |
| DSL1 | PR-0105 | CGCACCAACCTACAAGGGGGACTT | 22 |
| DSR4 | PR-0213 | ACAAAATGAAACGGCGGAGAGGT | 23 |
| DSR3 | PR-0212 | CCTTGTAGGTTGGTGCGAGGCACT | 24 |
| DSR8 | PR-377 | TGCTCCTCATACATTTGGTCAAAAACTGG | 25 |
| D2TIL1L | PR-0496 | TGAAGTTAGATTCCACAAGCTTGAAATTCG | 26 |
| D2TIL1R | PR-0498 | GAATTCCGTGCTGAGAATGATAAGTACACA | 27 |

The PCR amplification products may be screened for deoxyhypusine synthase mutations using any method that identifies nucleotide differences between wild type and mutant genes. These may include, for example but not limited to, sequencing, denaturing high pressure liquid chromatography (dHPLC), constant denaturant capillary electrophoresis (CDCE), temperature gradient capillary electrophoresis (TGCE) (Li et al., Electrophoresis, 23(10): 1499-1511,2002), or by fragmentation using enzymatic cleavage, such as used in the high throughput method described by Colbert et al., Plant Physiology, 126: 480-484, 2001. Preferably the PCR amplification products are incubated with an endonuclease that preferentially cleaves mismatches in heteroduplexes between wild type and mutant. Cleavage products are electrophoresed using an automated sequencing gel apparatus, and gel images are analyzed with the aid of a standard commercial image-processing program.

Mutations that reduce DHS function are desirable. Preferred mutations include missense and nonsense changes including mutations that prematurely truncate the translation of the DHS protein from messenger RNA, such as those mutations that create a stop codon within the coding region of the DHS gene. These mutations include point mutations, insertions, repeat sequences, and modified open reading frames (ORFs).

Once an M2 plant having a mutated deoxyhypusine synthase gene is identified, the mutations are analyzed to determine its affect on the expression, translation, and/or activity of the deoxyhypusine synthase enzyme. First, the PCR fragment containing the mutation is sequenced using standard sequencing techniques, in order to determine the exact location of the mutation within the deoxyhypusine synthase gene sequence. Each mutation is evaluated in order to predict its impact on protein function (i.e., completely tolerated to loss-of-function) using biofinormatics tools such as SIFT (Sorting Intolerant from Tolerant; Ng, P. C. and Henikoff, S. Nuc Acids Res 31: 3812-3814, 2003), PSSM (Position-Specific Scoring Matrix; Henikoff, J. G. and Henikoff, S. Comput Appl Biosci, 12: 135-143, 1996) and PARSESNP (Taylor N. E. and Greene, E. A. Nuc Acids Res, 31: 3808-3811, 2003). For example, a SIFT score that is less than 0.05 and a large change in PSSM score (roughly 10 or above) indicate a mutation that is likely to have a deleterious effect on protein function.

If the initial assessment of the mutation in the M2 plant indicated it to be of a useful nature and in a useful position within a deoxyhypusine synthase gene, then further phenotypic analysis of the tomato plant containing that mutation was pursued. First, the M2 plant was backcrossed or outcrossed twice to create a BC1 plant in order to eliminate background mutations. Then the backcrossed or outcrossed BC1 plant was self-pollinated in order to create a BC1F2 plant that was homozygous for the deoxyhypusine synthase mutation. If the mutation results in complete male sterility, the M2 plant cannot be self-pollinated in order to create a homozygous line. Therefore, the male sterile phenotype may be carried in a heterozygous state by crossing with pollinator or restorer lines having a wild type gene.

Deoxyhypusine synthase mutant tomatoes remain firm longer and display less skin wrinkling and rot post harvest than tomatoes from wild type sibling lines or or parental lines. The following mutations are exemplary of the tomato mutations created and identified according to the present invention. One exemplary mutation, is a G to A change at nucleotide 1064 of SEQ ID NO: 2. This mutation results in a change from glutamic acid at amino acid 87 in the expressed protein [SEQ ID NO: 3] to lysine.

Another exemplary mutation, is a C to T change at nucleotide 4202 of SEQ ID NO: 2. This mutation results in a change from proline at amino acid 256 in the expressed protein [SEQ ID NO: 3] to leucine.

Another exemplary mutation, is a G to A change at nucleotide 4538 of SEQ ID NO: 2. This mutation results in a change from glycine at amino acid 340 in the expressed protein [SEQ ID NO: 3] to arginine.

Another exemplary mutation is a G to A change at nucleotide 1091 of SEQ ID NO: 2. This mutation results in a change from valine at amino acid 96 in the expressed protein [SEQ ID NO: 3] to isoleucine.

Another exemplary mutation is a G to A change at nucleotide 539 of the partial genomic sequence designated as DHS2 (SEQ ID NO: 4) and nucleotide 400 of its corresponding coding sequence (SEQ ID NO: 5). This mutation results in a change to lysine from glutamic acid at amino acid 134 in the translated protein [SEQ ID NO: 6] corresponding to the coding sequence of the partial genomic sequence DHS2 [SEQ ID NO: 5].

The following Examples are offered by way of illustration, not limitation.

### EXAMPLE 1

### Mutagenesis

Tomato seeds of cultivars Shady Lady (hybrid) and NC 84173 (an inbred line provided by R. Gardner at UNC) were vacuum infiltrated in H₂O (approximately 1000 seeds/100 ml H₂O for approximately 4 minutes). The seeds were then placed on a shaker (45 rpm) in a fume hood at ambient temperature. The mutagen ethyl methanesulfonate (EMS) was added to the imbibing seeds to final concentrations ranging from about 0.1% to about 1.6% (v/v). EMS concentrations of about 0.4 to about 1.2% are preferable. Following a 24-hour incubation period, the EMS solution was replaced (4 times) with fresh H₂O. The seeds were then rinsed under running water for approximately 1 hour. Finally, the mutagenized seeds were planted (96/tray) in potting soil and allowed to germinate indoors. Plants that were four to six weeks old were transferred to the field to grow to fully mature M1 plants. The mature M1 plants were allowed to self-pollinate and then seeds from the M1 plant were collected and planted to produce M2 plants.

### DNA Preparation

DNA from these M2 plants was extracted and prepared in order to identify which M2 plants carried a mutation in their deoxyhypusine synthase gene. The M2 plant DNA was prepared using the methods and reagents contained in the Qiagen® (Valencia, Calif.) DNeasy® 96 Plant Kit. Approximately 50 mg of frozen plant sample was placed in a sample tube with a tungsten bead, frozen in liquid nitrogen and ground 2 times for 1 minute each at 20 Hz using the Retsch® Mixer Mill MM 300. Next 400 μl of solution AP1 [Buffer AP1, solution DX and RNAse (100 mg/ml)] at 80° C. was added to the sample. The tube was sealed and shaken for 15 seconds. Following the addition of 130 μl Buffer AP2, the tube was shaken for 15 seconds. The samples were placed in a freezer at minus 20° C. for at least 1 hour. The samples were then centrifuged for 20 minutes at 5600×g. A 400 μl aliquot of supernatant was transferred to another sample tube. Following the addition of 600 μl of Buffer AP3/E, this sample tube was capped and shaken for 15 seconds. A filter plate was placed on a square well block and 1 ml of the sample solution was applied to each well and the plate was sealed. The plate and block were centrifuged for 4 minutes at 5600×g. Next, 800 μl of Buffer AW was added to each well of the filter plate, sealed and spun for 15 minutes at 5600×g in the square well block. The filter plate was then placed on a new set of sample tubes and 80 μl of Buffer AE was applied to the filter. It was capped and incubated at room temperature for 1 minute and then spun for 2 minutes at 5600×g. This step was repeated with an additional 80 μl Buffer AE. The filter plate was removed and the tubes containing the pooled filtrates were capped. The individual samples were then normalized to a DNA concentration of 5 to 10 ng/μl.

### TILLING®

The M2 DNA was pooled into groups of four or more individuals each. For pools containing four individuals, the DNA concentration for each individual within the pool was 0.25 ng/μl with a final concentration of 1 ng/μl for the entire pool. The pooled DNA samples were arrayed on microtiter plates and subjected to gene-specific PCR.

PCR amplification was performed in 15 μl volumes containing 5ng pooled or individual DNA, 0.75X ExTaq buffer (Panvera®, Madison, Wis.), 2.6 mM MgCl₂, 0.3 mM dNTPs, 0.3 μM primers, and 0.05U Ex-Taq (Panvera®) DNA polymerase. PCR amplification was performed using an MJ Research® thermal cycler as follows: 95° C. for 2 minutes; 8 cycles of “touchdown PCR” (94° C. for 20 second, followed by annealing step starting at 70-68° C. for 30 seconds decreasing 1° C. per cycle, then a temperature ramp of 0.5° C. per second to 72° C. followed by 72° C. for 1 minute); 25-45 cycles of 94° C. for 20 seconds, 63-61° C. for 30 seconds, ramp 0.5° C./sec to 72° C., 72° C. for 1 minute; 72° C. for 8 minutes; 98° C. for 8 minutes; 80° C. for 20 seconds; 60 cycles of 80° C. for 7 seconds −0.3 degrees/cycle.

The PCR primers (MWG Biotech, Inc., High Point, N.C.) were mixed as follows:
9 μl 100 μM IRD-700 labeled left primer
1 μl 100 μM left primer
10 μl 100 μM right primer
   The IRD-700 label can be attached to either the right or left primer. Preferably, the labeled to unlabeled primer ratio is 9:1. Alternatively, Cy5.5 modified primers or IRD-800 modified primers could be used. The IRD-700 label was coupled to the nucleotide using conventional phosphoamidite chemistry.

PCR products (15 μl) were digested in 96-well plates. Next, 30 μl of a solution containing 10 mM HEPES [4-(2-hydroxyethyl)-1-piperazineethanesulfonic acid] (pH 7.5), 10 mM MgSO₄, 0.002% (w/v) Triton® X-100, 20 ng/ml of bovine serum albumin, and CEL 1 (Transgenomic®, Inc.; 1:100,000 dilution) was added with mixing on ice, and the plate was incubated at 45° C. for 15 min. The specific activity of the CEL1 was 800 units/μl, where a unit was defined by the manufacturer as the amount of enzyme required to produce 1 ng of acid-soluble material from sheared, heat denatured calf thymus DNA at pH 8.5 in one minute at 37° C. Reactions were stopped by addition of 10 μl of a 2.5 M NaCl solution with 0.5 mg/ml blue dextran and 75 mM EDTA, followed by the addition of 80 μl isopropanol. The reactions were precipitated at 80° C., spun at 4000 rpm for 30 minutes in an Eppendorf Centrifuge 5810. Pellets were resuspended in 8 μl of 33% formamide with 0.017% bromophenol blue dye, heated at 80° C. for 7 minutes and then at 95° C. for 2 minutes. Samples were transferred to a membrane comb using a comb-loading robot (MWG Biotech). The comb was inserted into a slab acrylamide gel (6.5%), electrophoresed for 10 min, and removed. Electrophoresis was continued for 4 h at 1,500-V, 40-W, and 40-mA limits at 50° C.

During electrophoresis, the gel was imaged using a LI-COR® (Lincoln, Nebr.) scanner which was set at a channel capable of detecting the IR Dye 700 label. The gel image showed sequence-specific pattern of background bands common to all 96 lanes. Rare events, such as mutations, create new bands that stand out above the background pattern. Plants with bands indicative of mutations of interest were evaluated by TILLING® individual members of a pool mixed with wild type DNA and then sequencing individual PCR products. Plants carrying mutations confirmed by sequencing were grown up as described above (e.g., the M2 plant was backcrossed or outcrossed twice in order to eliminate background mutations and self-pollinated in order to create a plant that was homozygous for the mutation).

### Shelf Life Determination

Tomatoes Selected for Study:

Individual tomatoes selected for study were harvested from plants derived from siblings of the same cross to preserve background phenotypes as much as possible. The plants and fruit were genotyped as homozygous for the mutation, heterozygous for the mutation, or wild type. Genotyping was performed using a genetic method for determining single base pair mismatches referred to in the scientific literature as “dCAPs,” see Neff et al., The Plant Journal 14: 387-392, 1998. Briefly, a degenerate PCR oligonucleotide was designed to create a restriction endonuclease recognition site when the mutant base pair is present. Plants were then simply genotyped using a PCR reaction followed by a restriction enzyme digestion and then analyzed on an agarose gel. In cases where wild type siblings were not available, tomatoes from the parental cultivar were used for comparison.

Shelf Life Assessment:

Greenhouse-grown tomato fruit was harvested at a variety of developmental stages including breaker, turning, pink and red. Tomatoes were stored at 18° C. or room temperature (approximately 24° C.) at ambient humidity and spaced one to three inches apart on shelves. Shelf life determinations were made for at least 30 to 60 days post harvest. Mutant tomatoes were always compared with controls (from either sibling or parental lines) that were harvested on the same day and matched for developmental stages. Fruit was photographed at regular intervals throughout the observation period. At the end of the observation period, the compiled photographs were used to rank the state of deterioration (less than, equal to, or greater than) of tomatoes carrying mutations in DHS compared to control tomatoes according to the degree of skin wrinkling, turgor loss and rot.

Tomatoes with mutations in DHS (including DHS1 mutants 1064, 4202, 4538, and 1091 and DHS2 mutant 539) displayed increased shelf life compared to matched control tomatoes regardless of the developmental stage at picking. For example, at 40 days post harvest, 40% of tomatoes that were homozygous for mutation DHS1 1091 were intact (not wrinkled or rotted) compared to only 20% of wild type sibling control tomatoes. At 60 days post harvest, 50% of tomatoes that were homozygous for mutation DHS2 539 were intact (not wrinkled or rotted) compared to only 29% of wild type sibling control tomatoes.

### Identification and Evaluation of DHS1 Mutation 1064

DNA from a tomato plant originating from seeds of cultivar Shady Lady that were incubated in 0.8% EMS, was amplified using primers DSLX and DSRX (SEQ ID NOs: 16 and 17). The PCR amplification products were then incubated with CEL 1 and electrophoresed. The electrophoresis gel image showed a fragment which stood out above the background pattern for the PCR amplification products. Therefore, it was likely that this fragment contained a heteroduplex created by a mutation in a deoxyhypusine synthase gene. Sequence analysis of this fragment showed the mutation was a G to A change at nucleotide 1064 of SEQ ID NO: 2. This mutation correlates with a change from glutamic acid to lysine at amino acid 87 of the deoxyhypusine synthase polypeptide shown in SEQ ID NO: 3.

### Identification and Evaluation of DHS1 Mutation 4202

DNA from a tomato plant originating from seeds of cultivar Shady Lady that were incubated in 0.8% EMS, was amplified using primers DSL3 and DSR5 (SEQ ID NOs: 18 and 19). The PCR amplification products were then incubated with CEL 1 and electrophoresed. The electrophoresis gel image showed a fragment which stood out above the background pattern for the PCR amplification products. Therefore, it was likely that this fragment contained a heteroduplex created by a mutation in a deoxyhypusine synthase gene. Sequence analysis of this fragment showed the mutation was a C to T change at nucleotide 4202 of SEQ ID NO: 2. This mutation correlates with a change from proline to leucine at amino acid 256 of the deoxyhypusine synthase polypeptide shown in SEQ ID NO: 3.

### Identification and Evaluation of DSH1 Mutation 4538

DNA from a tomato plant originating from seeds of cultivar Shady Lady that were incubated in 0.8% EMS, was amplified using primers DSL3 and DSR5 (SEQ ID NOs: 18 and 19). The PCR amplification products were then incubated with CEL 1 and electrophoresed. The electrophoresis gel image showed a fragment which stood out above the background pattern for the PCR amplification products. Therefore, it was likely that this fragment contained a heteroduplex created by a mutation in a deoxyhypusine synthase gene. Sequence analysis of this fragment showed the mutation was a G to A change at nucleotide 4538 of SEQ ID NO: 2. This mutation correlates with a change from glycine to arginine at amino acid 340 of the deoxyhypusine synthase polypeptide shown in SEQ ID NO: 3.

### Identification and Evaluation of DHS1 Mutation 1091

DNA from a tomato plant originating from seeds of cultivar Shady Lady that were incubated in 0.8% EMS, was amplified using primers DSLX and DSRX (SEQ ID NOs: 16 and 17). The PCR amplification products were then incubated with CEL 1 and electrophoresed. The electrophoresis gel image showed a fragment which stood out above the background pattern for the PCR amplification products. Therefore, it was likely that this fragment contained a heteroduplex created by a mutation in a deoxyhypusine synthase gene. Sequence analysis of this fragment showed the mutation was a G to A change at nucleotide 1091 of SEQ ID NO: 2. This mutation correlates with a change from valine to isoleucine at amino acid 96 of the deoxyhypusine synthase polypeptide shown in SEQ ID NO: 3.

### Identification and Evaluation of DHS2 Mutation 539

DNA from a tomato plant originating from seeds of cultivar Shady Lady that were incubated in 0.8% EMS, was amplified using primers D2TIL1L and D2TIL1R (SEQ ID NOs: 26 and 27). The PCR amplification products were then incubated with CEL 1 and electrophoresed. The electrophoresis gel image showed a fragment which stood out above the background pattern for the PCR amplification products. Therefore, it was likely that this fragment contained a heteroduplex created by a mutation in a deoxyhypusine synthase gene. Sequence analysis of this fragment showed the mutation was a G to A change at nucleotide 539 of the partial genomic sequence designated as DHS2 (SEQ ID NO: 4) and nucleotide 400 of its corresponding coding sequence (SEQ ID NO: 5). This mutation correlates with a change to lysine from glutamic acid at amino acid 134 of the translated protein [SEQ ID NO: 6] corresponding to the coding sequence of the partial genomic sequence DHS2 [SEQ ID NO: 5].

The above examples are provided to illustrate the invention but not limit its scope. Other variants of the invention will be readily apparent to one of ordinary skill in the art and are encompassed by the appended claims and all their equivalents. All publications, patents, and patent applications cited herein are hereby incorporated by reference.

1. A tomato plant, tomato fruits, seeds, plant parts and progeny thereof having reduced deoxyhypusine synthase activity compared to the wild type tomato plants wherein the reduced deoxyhypusine synthase activity is caused by a non-transgenic mutation in a deoxyhypusine synthase gene.

2. Food and food products incorporating a tomato fruit of claim 1.

3. Pollen from the tomato plant of claim 1.

4. The tomato plant of claim 1 having tomato fruits that have delayed spoilage compared to wild type tomato fruits due to reduced deoxyhypusine synthase activity.

5. The tomato plant of claim 4 wherein the delayed spoilage is due to a slower softening of the tomato fruits.

6. The tomato plant of claim 4 wherein the delayed spoilage causes an increase in shelf life of the harvested tomatoes.

7. Tomato fruits, seeds, plant parts and progeny of the tomato plant of claim 4.

8. Pollen from the tomato plant of claim 4.

9. Food and food products incorporating a tomato fruit of claim 4.

10. An endogenous deoxyhypusine synthase gene having substantial homology to SEQ. I.D. No. 2 and having a non-transgenic mutation within the endogenous deoxyhypusine synthase gene.

11. The endogenous deoxyhypusine synthase gene of claim 10 wherein the non-transgenic mutation occurs around nucleotide 1064 of SEQ. I.D. No. 2.

12. A tomato plant containing the endogenous deoxyhypusine synthase gene of claim 11.

13. Tomato fruits, seeds, pollen, plant parts, and progeny of the tomato plant of claim 12.

14. Food and food products incorporating the fruit of the tomato plant of claim 12.

15. The endogenous deoxyhypusine synthase gene of claim 10 wherein the non-transgenic mutation creates a change in at least amino acid 87 of the deoxyhypusine synthase protein expressed from the deoxyhypusine synthase gene.

16. A deoxyhypusine synthase protein expressed from the endogenous deoxyhypusine synthase gene of claim 15.

17. The deoxyhypusine synthase of claim 16 having a lysine at amino acid 87.

18. The endogenous deoxyhypusine synthase gene of claim 10 wherein the non-transgenic mutation occurs around nucleotide 4202 of SEQ. I.D. No. 2.

19. A tomato plant containing the endogenous deoxyhypusine synthase gene of claim 18.

20. Tomato fruits, seeds, pollen, plant parts, and progeny of the tomato plant of claim 19.

21. Food and food products incorporating the fruit of the tomato plant of claim 19.

22. The endogenous deoxyhypusine synthase gene of claim 10 wherein the non-transgenic mutation creates a change in at least amino acid 256 of the deoxyhypusine synthase protein expressed from the deoxyhypusine synthase gene.

23. A deoxyhypusine synthase protein expressed from the endogenous deoxyhypusine synthase gene of claim 22.

24. The deoxyhypusine synthase of claim 23 having a leucine at amino acid 256.

25. The endogenous deoxyhypusine synthase gene of claim 10 wherein the non-transgenic mutation occurs around nucleotide 4538 of SEQ. I.D. No. 2.

26. A tomato plant containing the endogenous deoxyhypusine synthase gene of claim 25.

27. Tomato fruits, seeds, pollen, plant parts, and progeny of the tomato plant of claim 26.

28. Food and food products incorporating the fruit of the tomato plant of claim 26.

29. The endogenous deoxyhypusine synthase gene of claim 10 wherein the non-transgenic mutation creates a change in at least amino acid 340 of the deoxyhypusine synthase protein expressed from the deoxyhypusine synthase gene.

30. A deoxyhypusine synthase protein expressed from the endogenous deoxyhypusine synthase gene of claim 29.

31. The deoxyhypusine synthase of claim 29 having an arginine at amino acid 340.

32. The endogenous deoxyhypusine synthase gene of claim 10 wherein the non-transgenic mutation occurs around nucleotide 1091 of SEQ. I.D. No. 2.

33. A tomato plant containing the endogenous deoxyhypusine synthase gene of claim 30.

34. Tomato fruits, seeds, pollen, plant parts, and progeny of the tomato plant of claim 33.

35. Food and food products incorporating the fruit of the tomato plant of claim 33.

36. The endogenous deoxyhypusine synthase gene of claim 10 wherein the non-transgenic mutation creates a change in at least amino acid 96 of the deoxyhypusine synthase protein expressed from the deoxyhypusine synthase gene.

37. A deoxyhypusine synthase protein expressed from the endogenous deoxyhypusine synthase gene of claim 36.

38. The deoxyhypusine synthase of claim 36 having an isoleucine at amino acid 96.

39. An endogenous deoxyhypusine synthase gene having substantial homology to SEQ. I.D. No. 4.

40. The endogenous deoxyhypusine synthase gene of claim 39 having a non-transgenic mutation within the endogenous deoxyhypusine synthase gene.

41. A tomato plant containing the endogenous deoxyhypusine synthase gene of claim 40.

42. Tomato fruits, seeds, pollen, plant parts, and progeny of the tomato plant of claim 41.

43. Food and food products incorporating the fruit of the tomato plant of claim 41.

44. The endogenous deoxyhypusine synthase gene of claim 40 wherein the non-transgenic mutation occurs around nucleotide 539 of SEQ. I.D. No. 4.

45. A tomato plant containing the endogenous deoxyhypusine synthase gene of claim 44.

46. Tomato fruits, seeds, pollen, plant parts, and progeny of the tomato plant of claim 45.

47. Food and food products incorporating the fruit of the tomato plant of claim 45.

48. The endogenous deoxyhypusine synthase gene of claim 48 wherein the non-transgenic mutation creates a change in at least amino acid 134 of the deoxyhypusine synthase protein translated from the deoxyhypusine synthase sequence of SEQ ID NO: 4.

49. A deoxyhypusine synthase protein expressed from the endogenous deoxyhypusine synthase gene of claim 48.

50. The deoxyhypusine synthase protein of claim 49 having a lysine at amino acid 134.

51. An isolated nucleic acid construct comprising a deoxyhypusine synthase polynucleotide sequence having substantial homology to SEQ. I.D. Nos. 4 or 5.

52. An isolated nucleic acid construct comprising a deoxyhypusine synthase polynucleotide sequence complementary to SEQ. I.D. Nos. 4 or 5.

53. The nucleic acid construct of claim 55 wherein the deoxyhypusine synthase polynucleotide sequence encodes a deoxyhypusine synthase polypeptide having substantial similarity to SEQ. I.D. No. 6.

54. A method of creating a tomato plant exhibiting reduced deoxyhypusine synthase enzyme activity compared to wild type tomato plants, comprising the steps of:
a. obtaining plant material from a parent tomato plant;
b. inducing at least one mutation in at least one copy of a deoxyhypusine synthase gene of the plant material by treating the plant material with a mutagen to create mutagenized plant material;
c. culturing the mutagenized plant material to produce progeny tomato plants;
d. analyzing progeny tomato plants to detect at least one mutation in at least one copy of a deoxyhypusine synthase gene;
e. selecting progeny tomato plants that have reduced deoxyhypusine synthase enzyme activity compared to a wild type tomato plant; and
f. repeating the cycle of culturing the progeny tomato plants to produce additional progeny tomato plants having reduced deoxyhypusine synthase enzyme activity.

55. The method of claim 54 wherein the plant material is selected from the group consisting of seeds, pollen, plant cells, or plant tissue.

56. The method of claim 54 wherein the mutagen is ethyl methanesulfonate.

57. The method of claim 56 wherein the concentration of ethyl methanesulfonate used is from about 0.4 to about 1.2%.

58. The method of claim 54 wherein the steps to create the tomato plant further included analyzing the progeny tomato plants by
a. isolating genomic DNA from the progeny tomato plants; and
b. amplifying segments of a deoxyhypusine synthase gene in the isolated genomic DNA using primers specific to a deoxyhypusine synthase gene or to the DNA sequences adjacent to a deoxyhypusine synthase gene.

59. The method of claim 58 wherein at least one primer has a sequence substantially homologous to a sequence as shown in the group consisting of SEQ. I.D. Nos. 16 through 27.

60. The method of claim 54 wherein the mutation detected in step d is evaluated to determine the mutations likelihood of having a deleterious effect on deoxyhypusine synthase enzyme activity.

61. The method of claim 60 wherein the mutation is evaluated using a bioinformatics tool selected from the group consisting of SIFT, DSSM, and PARSESNP.

62. A tomato plant created according to the method of claim 54.

63. Tomato fruit, seeds, pollen, plant parts or progeny of the tomato plant of claim 62.

64. Food and food products incorporating the fruit of a tomato plant of claim 62.


SEQUENCE LISTING (27 sequences)

SEQ ID NO: 1 - DNA, 1610 bp, Lycopersicon esculentum
cgcagaaactcgcggcggcagtcttgttccgtacataatcttggtctgcaataatgggagaagctctgaagtacagtatcatggactcagtaagatcggtagttttcaaagaatccgaaaatctagaaggttcttgcactaaaatcgagggctacgacttcaataaaggcgttaactatgctgagctgatcaagtccatggtttccactggtttccaagcatctaatcttggtgacgccattgcaattgttaatcaaatgctagattggaggctttcacatgagctgcccacggaggattgcagtgaagaagaaagagatgttgcatacagagagtcggtaacctgcaaaatcttcttggggttcacttcaaaccttgtttcttctggtgttagagacactgtccgctaccttgttcagcaccggatggttgatgttgtggttactacagctggtggtattgaagaggatctcataaagtgcctcgcaccaacctacaagggggacttctctttacctggagcttctctacgatcgaaaggattgaaccgtattggtaacttattggttcctaatgacaactactgcaaatttgagaattggatcatcccagtttttgaccaaatgtatgaggagcagattaatgagaaggttctatggacaccatctaaagtcattgctcgtctgggtaaagaaattaatgatgaaacctcatacttgtattgggcttacaagaaccggattcctgtcttctgtcctggcttgacggatggatcacttggtgacatgctatacttccattctttcaaaaagggtgatccagataatccagatcttaatcctggtctagtcatagacattgtaggagatattagggccatgaatggtgaagctgtccatgctggtttgaggaagacaggaatgattatactgggtggagggctgcctaagcaccatgtttgcaatgccaatatgatgcgcaatggtgcagattttgccgtcttcattaacaccgcacaagagtttgatggtagtgactctggtgcccgtcctgatgaagctgtatcatggggaaagatacgtggtggtgccaagactgtgaaggtgcattgtgatgcaaccattgcatttcccatattagtagctgagacatttgcagctaagagtaaggaattctcccagataaggtgccaagtttgaacattgaggaagctgtccttccgaccacacatatgaattgctagcttttgaagccaacttgctagtgtgcagcaccatttattctgcaaaactgactagagagcagggtatattcctctaccccgagttagacgacatcctgtatggttcaaattaattatttttctccccttcacaccatgttatttagttctcttcctcttcgaaagtgaagagcttagatgttcataggttttgaattatgttggaggttggtgataactgactagtcctcttaccatatagataatgtatccttgtactatgagattttgggtgtgtttgataccaaggaaaaatgtttatttggaaaacaattggatttttaatttattttttcttgtttaaaaaaaaaaaaaaaaaaaaaaaaaa

SEQ ID NO: 2 - DNA, 4755 bp, Lycopersicon esculentum
atgggagaagctctgaagtacagtatcatggactcagtaagatcggtagttttcaaagaatccgaaaatctagaaggttcttgcactaaaatcgagggctacgacttcaataaaggcgttaactatgctgagctgatcaagtccatggtttccactggtttccaagcatctaatcttggtgacgccattgcaattgttaatcaaatggtcagattcacttctttcttttaatttacgctaaaaaatttgttgctttatgcaaattttcatatttccgtatcaagattttttttccagctcctatgatattgaatttttttcacctcatccgcatatttttttgggcttagtaataaagttaattgctttacaccgacaatatttgggtttcaactactctctccttcccattttatatgactggaaacagagtttaagatgttagattgacttaatattagtatgtcattagtcttggtagagaaattatcacaatgaagttttattttgctgtaattaatttggattctaaaagatcatgaaagttgtatagatatggaataatatcagtcatgttttggaagtcatgaaatatagcaagtagcagtggaacggtgaaagttaaaaatttagagttgatagaatagttttgtaggcaaagtatgttaaaagaattaatcctaaaatagaaagaatgctaattaagttggaattggtgggtatagatagtatgattcatggtacttgtatgcaaattggaagtatgtaagacttatttcaattcaattaatatctgaatttacttcggaaaatctgttcaccctcacaagttaaacctcaccgatcttttaacagttgatgatagtgcttcttaaaaaatgttttgactttttataaacaggttacgttctttcatacacttggaagttgttgaactagttttgaagttcttaaaattgattctagaagtttgaaatttgtgtttttgtttgggggtgtgatgggtttgaagctagattggaggctttcacatgagctgcccacggaggattgcagtgaagaagaaagagatgttgcatacagagagtcggtaacctgcaaaatcttcttggggttcacttcaaaccttgtttcttctggtgttagagacactgtccgctaccttgttcagcaccggatggtgagttgcatttaagtaacagttccagtcgtttctgttctactaattcttttaagtctgactgtgttgtaccattaagtttgaatcttgtgggcttcttttatttgagatcatcttaatatatcttctcaatttttttatgtcctctccgtgtacactctaaagctatacttaccacactaatactatgacatcctcttattcaacctcaatactcttcatatgatttattatgtcaagcactgaatcgctgctatgcttggtttgtattaattgaagatcaagccttttgacattatttctcatgttccctatattccttgtctctctgatttattggttacaaaaactagaatacctcatccttctatctgccacacaagtactaaatgatatggtagaaaattattatctttgcgtcttagatttcagctctcatgtttagcattggtctaaagaaacttttgttaccatatggaacaattttctattaattattgtgtcttggaccttagcaaactgtcttccctccctgcaggttgatgttgtggttactacagctggtggtattgaagaggatctcataaagtgcctcgcaccaacctacaagggggacttctctttacctggagcttctctacgatcgaaaggattgaaccgtattggtaacttattggttcctaatgacaactactgcaaatttgagaattggatcatcccagtttttgaccaaatgtatgaggagcagattaatgaggtatccgctcttgtctgattcagtgttagatcatcccattacttgtttaatattggctgaagctttaacattcaatctggattctgctattactttcaaatacaaatggattttttacttgggtcattgcgtttgtgatattctttcaaatgtgataatacttatcttagctttggcttgtgatgatttgtttatcttctttttgtgatacagaaggttctatggacaccatctaaagtcattgctcgtctgggtaaagaaattaatgatgaaacctcatacttgtattgggcttacaaggtacatttctaatcacttccctttcattacatatttgatgaaattgtaatactcaagttagaatgccaaatctgtcccacatatataatagttacctctccgccgtttcattttgtaaggtgcttttcaaatcacttcccttccgttccatatttgatgaaattaaattggtgaagttagaatgccaaatgatctagctagtcccacatatataattaattattagttacgacttcccctttccattttattggcatagtttgattggacatgaagtttataactgtaaagaatactgttgaatcttgtgtcataaactaaattttgtatatcatgccaaaaatatccgttgaatcttgtgctcttaaatatgtcatgtcatttatgtaagtgagtggggagtgttattaagggtatgttgaagttaaaaacttactaaatatagaaagagacattctttttagacaaactaaaacgagaaggcacataaattgaaatggagggagtaatattgggatgatattttgataaatgagaaagtaagagatatttacctgggagaaaatggatttgcatgcattgtggtagcaaattcatcgttattgagatcagaaagtgaaatcaagagggaaggataaaaggagaagaaaccttcatttttttatttttttttgaaataatgaggattcatctagctgaccccatcccgtttgtgactgaggcgtagctgttattttcctatattttttttgtttagacgccatagaaccccttaaactatttcctttgaattcaagaaagaaaaagaagtacaacattatataggaaggaaaaagaaaagtttaaaaaaggatggatacaacttcagaattctggcagatttttgcaatacttaatttggaggatatcttttgtgcagaaaacatcaataacagttttttctcttatcagatcctttctgcttgccaaatgcacaaaacttcaggaaagtagcttctatcattattcccaggttttccttgcaattaagtagtacctttgtcattctaggtactaaacgttgttatagttttcgtctttcaggtgggaataatagggatagggttgaggatatcttccggaacacccagctgttgttttctggaaaatattgacttttacctagcaaactttacattagtactcaatagggaagaacagaataggataggtctctttccaaagggggggggtatgaattgaaacacaataccttattttttggatggaaaattaagtcatcctagttaatcttattattattattttggtttttggactcggctgatgttgtaaatagttcagcaatctatcttagtgctgttttgacaaactgaaaatcctattaccttctcaaaaataaaaaggatgtagaaatacctgttttatctctagatattccaagtgaagttttttcacaagcagaaatattttgaatttctaatagaataaatcttgtttccatgaagtttctaaagtttgcccctacaactttatttttttctttcaatccggaatttgctgtagttgcacgttggggtgtgataccagaatactagacaatgggatgaactaaggcatgatctcactattatccattccactatactgatttaaagcccagcgattgacatattatgataatttttggatgatcagaattcagaggttcatcgtggaaattttttgaatttttacttgggcagtttctcgggaagtcatattcactctattatgaagaagggatgatttcatgctttttcagcccagaaaataaaaggaaaacatgcattctcttttagttcatatattaatgttactctcgtatttatcatctcagaaccggattcctgtcttctgtcctggcttgacggatggatcacttggtgacatgctatacttccattctttcaaaaagggtgatccagataatccagatcttaatcctggtctagtcatagacattgtaggaggtatgagtatcttatcaagtgattctttcctctcatcaagaatttgtttgatgaaagagctaacatgattaagttatataaccagatattagggccatgaatggtgaagctgtccatgctggtttgaggaagacaggaatgattatactgggtggagggctgcctaagcaccatgtttgcaatgccaatatgatgcgcaatggtgcagattttgccgtcttcattaacaccgcacaagagtttgatggtagtgactctggtgcccgtcctgatgaagctgtatcatggggaaagatacgtggtggtgccaagactgtgaaggttaacaacttacctctaatacattttgtgcaagcatctcctttgtgagattctaatttgttgtagtgtttttgatttgttgaatgcaggtgcattgtgatgcaaccattgcatttcccatattagtagctgagacatttgcagctaagagtaaggaattctcccagataaggtgccaagtttga

SEQ ID NO: 3 - protein, 381 aa, Lycopersicon esculentum
MGEALKYSIMDSVRSVVFKESENLEGSCTKIEGYDFNKGVNYAELIKSMVSTGFQASNLGDAIAIVNQMLDWRLSHELPTEDCSEEERDVAYRESVTCKIFLGFTSNLVSSGVRDTVRYLVQHRMVDVVVTTAGGIEEDLIKCLAPTYKGDFSLPGASLRSKGLNRIGNLLVPNDNYCKFENWIIPVFDQMYEEQINEKVLWTPSKVIARLGKEINDETSYLYWAYKNRIPVFCPGLTDGSLGDMLYFHSFKKGDPDNPDLNPGLVIDIVGDIRAMNGEAVHAGLRKTGMIILGGGLPKHHVCNANMMRNGADFAVFINTAQEFDGSDSGARPDEAVSWGKIRGGAKTVKVHCDATIAFPILVAETFAAKSKEFSQIRCQV

SEQ ID NO: 4 - DNA, 1241 bp, Lycopersicon esculentum
tccatgatttccactggtttccaagcttctaatcttggtgacgccattgacattgttaatcaaatggttagatttcacttttttttttaagattgaagttagattccacaagcttgaaattcgtgtttttgaagctaaattggaggctttcacacgaggtgcccacggaggattgcagcgcaagagaaagagatattgcatatagagagtcgataacgtgcaaaatatttttggggttcacttcaaaccttgtttcttctggtgttagagagaccattcgttaccttgttcagcaccgtatggtcagtaccatttccattactttatgtctctattaccgtcaagtttgaaccttagtgttccctccctacaggttgacgttttggttactacagctggtggtattgaagaggatctcataaaatgcctcgcaccgacttataagggggactttgctttacctggagctgctctacggtccaaagcattgaaccgtattggtaatttattggttccaaatgataactactgcaaatttgaggattggatcatgccaatttttgaccaaatgtatgacgagcagactaatcaggtacctatctagtactcttggctcattaattaattgcgtttctgtgtgtcattatttagcttctttttgtgttacagaacgttctatggacaccatctaaagtgattgctcgacttggcaaagagattaatgatgaaacctcatacttgtattgggcttacaaggtttgttattagtgcttatttaatttgattctgagtctataaactaatgtgtacttatcattctcagcacggaattcctgtcttctgtcctggcttgacggatggatcaataggtgacatgctatattttcattcttaccaaaagggagaccaagatcgtaatcctggtctagtgttagacattgttggaggtatcatatcactatcttatcgatcgatcaagtgattctaattctttcatcatgaatttgtttgacaataaagaattatataaccagatgtgagggcaatggataatgaagctgtccatgctggtttgaggaagacaggaataattatcttgggcggaggactgcctaagcaccatatttgcaatgcaaatatgatgcgcaatggtgcagattttgctgtcttaattaacactgcacaggagtatgatggtagtgactccggtgcccgtcctgatgaagctgtatcatggggaa

SEQ ID NO: 5 - DNA, 871 bp, Lycopersicon esculentum
tccatgatttccactggtttccaagcttctaatcttggtgacgccattgacattgttaatcaaatgctaaattggaggctttcacacgaggtgcccacggaggattgcagcgcaagagaaagagatattgcatatagagagtcgataacgtgcaaaatatttttggggttcacttcaaaccttgtttcttctggtgttagagagaccattcgttaccttgttcagcaccgtatggttgacgttttggttactacagctggtggtattgaagaggatctcataaaatgcctcgcaccgacttataagggggactttgctttacctggagctgctctacggtccaaagcattgaaccgtattggtaatttattggttccaaatgataactactgcaaatttgaggattggatcatgccaatttttgaccaaatgtatgacgagcagactaatcagaacgttctatggacaccatctaaagtgattgctcgacttggcaaagagattaatgatgaaacctcatacttgtattgggcttacaagcacggaattcctgtcttctgtcctggcttgacggatggatcaataggtgacatgctatattttcattcttaccaaaagggagaccaagatcgtaatcctggtctagtgttagacattgttggagatgtgagggcaatggataatgaagctgtccatgctggtttgaggaagacaggaataattatcttgggcggaggactgcctaagcaccatatttgcaatgcaaatatgatgcgcaatggtgcagattttgctgtcttaattaacactgcacaggagtatgatggtagtgactccggtgcccgtcctgatgaagctgtatcatggggaa

SEQ ID NO: 6 - protein, 290 aa, Lycopersicon esculentum
SMISTGFQASNLGDAIDIVNQMLNWRLSHEVPTEDCSARERDIAYRESITCKIFLGFTSNLVSSGVRETIRYLVQHRMVDVLVTTAGGIEEDLIKCLAPTYKGDFALPGAALRSKALNRIGNLLVPNDNYCKFEDWIMPIFDQMYDEQTNQNVLWTPSKVIARLGKEINDETSYLYWAYKHGIPVFCPGLTDGSIGDMLYFHSYQKGDQDRNPGLVLDIVGDVRAMDNEAVHAGLRKTGIIILGGGLPKHHICNANMMRNGADFAVLINTAQEYDGSDSGARPDEAVSWG

SEQ ID NO: 7 - DNA, 21 bp, Artificial
  Primer DSL0
cggcggcagtcttgttccgta

SEQ ID NO: 8 - DNA, 25 bp, Artificial
  Primer DSseqL
ttggattctaaaagatcatgaaagt

SEQ ID NO: 9 - DNA, 24 bp, Artificial
  Primer DS-L1
cgcaccaacctacaagggggactt

SEQ ID NO: 10 - DNA, 22 bp, Artificial
  Primer DSL2
aaatcacttcccttccgttcca

SEQ ID NO: 11 - DNA, 23 bp, Artificial
  Primer DSL3
ttgggcagtttctcgggaagtca

SEQ ID NO: 12 - DNA, 24 bp, Artificial
  Primer DS-R1
gtgatccatccgtcaagccaggac

SEQ ID NO: 13 - DNA, 23 bp, Artificial
  Primer DSR2
accccaacgtgcaactacagcaa

SEQ ID NO: 14 - DNA, 24 bp, Artificial
  Primer DSR3
ccttgtaggttggtgcgaggcact

SEQ ID NO: 15 - DNA, 24 bp, Artificial
  Primer DSR5
tggtcggaaggacagcttcctcaa

SEQ ID NO: 16 - DNA, 26 bp, Artificial
  Primer DSLX
tcggaaaatctgttcaccctcacaag

SEQ ID NO: 17 - DNA, 26 bp, Artificial
  Primer DSRX
gcatagcagcgattcagtgcttgaca

SEQ ID NO: 18 - DNA, 23 bp, Artificial
  Primer DSL3
ttgggcagtttctcgggaagtca

SEQ ID NO: 19 - DNA, 24 bp, Artificial
  Primer DSL5
tggtcggaaggacagcttcctcaa

SEQ ID NO: 20 - DNA, 21 bp, Artificial
  Primer DSL0
cggcggcagtcttgttccgta

SEQ ID NO: 21 - DNA, 29 bp, Artificial
  DS-R6
tgaaacccaaatattgtcggtgtaaagca

SEQ ID NO: 22 - DNA, 24 bp, Artificial
  Primer DSL1
cgcaccaacctacaagggggactt

SEQ ID NO: 23 - DNA, 23 bp, Artificial
  Primer DSR4
acaaaatgaaacggcggagaggt

SEQ ID NO: 24 - DNA, 24 bp, Artificial
  Primer DSR3
ccttgtaggttggtgcgaggcact

SEQ ID NO: 25 - DNA, 29 bp, Artificial
  Primer DSR8
tgctcctcatacatttggtcaaaaactgg

SEQ ID NO: 26 - DNA, 30 bp, Artificial
  Primer D2TIL1L
tgaagttagattccacaagcttgaaattcg

SEQ ID NO: 27 - DNA, 30 bp, Artificial
  Primer D2TIL1R
gaattccgtgctgagaatgataagtacaca